(19) United States
(12) Patent Application Publication
(10) Pub. No.: US 2026/0055154 A1
(43) Pub. Date: Feb. 26, 2026

(54) LONG-ACTING DUAL-AGONIST COMPOUND

(71) Applicant: CHENGDU AODA BIOTECHNOLOGY CO., LTD, Chengdu (CN)

(72) Inventors: Shuliang ZHOU, Chengdu (CN); Peng WANG, Chengdu (CN); Lan DENG, Chengdu (CN)

(21) Appl. No.: 19/102,413

(22) PCT Filed: Aug. 7, 2023
(86) PCT No.: PCT/CN2023/111384

§ 371 Date: Feb. 10, 2025

(30) Foreign Application Priority Data
Aug. 10, 2022 (CN) 202210957875.5

(51) Int. Cl. C07K 14/605; A61K 38/26

(52) U.S. Cl. CPC C07K 14/605; A61K 38/26

(57) ABSTRACT

The present invention relates to the field of pharmaceutical synthesis. A GLP-1/Gcg dual-agonist compound is disclosed. The GLP-1/Gcg dual-agonist compound is used for preparing a pharmaceutical composition for treating disease. Use of the pharmaceutical composition in the preparation of a drug for treating at least one of the following diseases: type-II diabetes, impaired glucose tolerance, type-I diabetes, obesity, hypertension, metabolic syndrome, dyslipidemia, cognitive disorders, atherosclerosis, myocardial infarction, coronary heart disease, cardiovascular disease, strokes, inflammatory bowel syndrome and/or dyspepsia or gastric ulcers, hepatic fibrosis disease, and pulmonary fibrosis disease.

### CROSS REFERENCE TO RELATED APPLICATIONS

This application is a US National Phase application based upon PCT Application No. PCT/CN2023/111384 filed Aug. 7, 2023, which claims the priority of Chinese Patent Application No. 202210957875.5 filed with the China National Intellectual Property Administration on Aug. 10, 2022, and titled with “LONG-ACTING DUAL-AGONIST COMPOUND”, the disclosure of which is hereby incorporated by reference in its entirety.

### SEQUENCE LISTING

This application contains references to amino acid sequences and/or nucleic acid sequences which have been submitted concurrently herewith as the sequence listing text file entitled “OP2423-US-0813 Sequence Listing”, file size 11,700 bytes, created on Jan. 26, 2025. The aforementioned sequence listing is hereby incorporated by reference in its entirety pursuant to 37 C.F.R. § 1.52 (e)(5).

### FIELD

The present disclosure relates to the field of pharmaceutical synthesis, in particular to a long-acting dual-agonist compound, which is a dual-agonist compound of glucagon (Gcg) receptor and glucagon-like peptide-1 (GLP-1) receptor.

### BACKGROUND

GLP-1 is a 37-amino-acid peptide that stimulates insulin secretion, protects pancreatic beta cells, and inhibits glucagon secretion, gastric emptying and food intake, leading to weight loss. GLP-1 is known as an incretin. Incretin receptor signaling plays a key physiologically relevant role in glucose homeostasis. In normal physiology, GLP-1 is secreted from the intestine after a meal. These incretins enhance physiological responses to food, including satiety, insulin secretion, and nutrient disposal.

The most common side effect of GLP-1 analogs is that the administration thereof does not achieve full-effect blood sugar control and weight loss, while GIP alone has a very modest glucose-lowering ability in patients with type 2 diabetes. Natural GLP-1 can be rapidly inactivated by the ubiquitous protease DPP IV and can therefore only be used for short-term metabolic control.

Geg, also known as glucagon or anti-insulin or insulin B, is a hormone secreted by the pancreatic islet alpha cells of the vertebrate pancreas along with insulin, which antagonizes insulin and plays a role in increasing blood sugar.

New studies have shown that dual GLP-1 and Geg receptor agonists not only have better blood sugar control, but also have the effect of significantly reducing weight and treating non-alcoholic fatty liver disease.

Currently, there is no dual GLP-1/Gcg receptor agonist on the market. The half-life of the dual GLP-1/Gcg receptor agonist OXM3 in clinical research is 120 hrs, and the administration cycle is once a week.

### SUMMARY

In view of this, the present disclosure provides a long-acting dual agonist compound, which is a dual agonist compound of glucagon (Gcg) receptor and glucagon-like peptide-1 (GLP-1) receptor.

In order to achieve the above-mentioned disclosure purpose, the present disclosure provides the following embodiments.

The present disclosure provides a compound, including:
(I) an amino acid sequence set forth in Formula I,

Formula I
(SEQ ID NO: 6)
His-AA1-Gln-Gly-Thr-Phe-Thr-Ser-Asp-Tyr-Ser-Lys-
Tyr-Leu-Asp-Glu-Lys-Lys-Ala-Lys(R)-Glu-Phe-Val-
Glu-Trp-Leu-Leu-AA2-Gly-Gly-Pro-Ser-Ser-Gly-Ala-
Pro-Pro-Pro-Ser-AA3

AA1 in Formula I is selected from the group consisting of Aib, Acpr, Acp, Acpe and Ach;
AA2 in Formula I is selected from the group consisting of Glu and Ser;
AA3 in Formula I is selected from the group consisting of NH₂ and OH;
R in Formula I is selected from the group consisting of

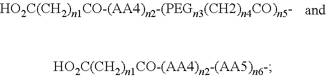

In one embodiment, AA4 is selected from the group consisting of γGlu, εLys, β-Ala, γ-aminobutyric acid and 5-Ava; and
AA5 is selected from the group consisting of Gly, Ser, Thr, Asp, Glu, Aad, Lys, Orn, Dab and Dap; or
(II) an amino acid sequence obtained from substitution, deletion, addition and/or replacement of one or more amino acids of the amino acid sequence set forth in (I); or
(III) a sequence with more than 90% homology to the amino acid sequence set forth in (I); or
(IV) a pharmaceutically acceptable salt, solvate, chelate or non-covalent complex of the compound shown in Formula I; and/or
(V) a drug precursor based on the compound shown in Formula I; and/or
(VI) a mixture including (I), (II), (III), (IV) and/or (V).

In some specific embodiments of the present disclosure,
n1 is an integer selected from 10 to 20;
n2 is an integer selected from 1 to 5;
n3 is an integer selected from 1 to 30;
n4 is an integer selected from 1 to 5;
n5 is an integer selected from 1 to 10; and
n6 is an integer selected from 1 to 10.

The present disclosure further provides a method for producing the compound, including:
Step 1, performing solid-phase polypeptide synthesis to obtain a peptide resin; and
Step 2, performing acid hydrolysis and purification to obtain the compound.

The present disclosure further provides use of any one of the following in the manufacture of a dual GLP-1 receptor and Gcg receptor agonist:
(I) the compound; and/or
(II) the compound produced by the method.

The present disclosure further provides use of any one of the following in the manufacture of a medicament for preventing and/or treating a disease:
(I) the compound; and/or
(II) the compound produced by the method.

In some specific embodiments of the present disclosure, the disease includes: type II diabetes, impaired glucose tolerance, type I diabetes, obesity, hypertension, metabolic syndrome, dyslipidemia, cognitive impairment, atherosclerosis, myocardial infarction, coronary heart disease, cardiovascular disease, stroke, inflammatory bowel syndrome and/or dyspepsia or gastric ulcer, liver fibrosis and/or pulmonary fibrosis.

The present disclosure further provides use of any one of the following in the manufacture of a drug and/or drug combination for treating type II diabetes with long-lasting efficacy and/or preventing deterioration of type II diabetes:
(I) the compound; and/or
(II) the compound produced by the method.

The present disclosure further provides use of the compound or the compound produced by the method in the manufacture of a drug and/or a drug combination for regulating blood sugar in the body;

In one embodiment, the regulation of blood sugar in the body includes reducing food intake, reducing β cell apoptosis, increasing pancreatic β cell function, increasing β-cell mass and/or restoring the sensitivity of β-cell to glucose.

The present disclosure further provides a drug or drug combination, including any one of the following:
(I) the compound; and/or
(II) the compound produced by the method.

The present disclosure further provides a method for regulating blood sugar in the body, including administering any one of the following to a subject in need thereof:
(I) the compound; and/or
(II) the compound produced by the method; and/or
(III) the drug or drug combination.

The present disclosure includes but is not limited to the following beneficial effects.

The compound shown in Formula I provided by the present disclosure is a dual Gcg/GLP-I receptor agonist analog, which has the biological activity of lowering blood sugar and reducing body weight.

### DETAILED DESCRIPTION

The present disclosure discloses a long-acting dual agonist compound, and can refer to the content herein and appropriately improve the process parameters to achieve it. It should be particularly noted that all similar substitutions and modifications may be made, which are all considered to be included in the present disclosure. The method and application of the present disclosure have been described through some embodiments, and relevant personnel can be modify or appropriately change and combine the methods and applications described herein without departing from the content, embodiments of the present disclosure to achieve and apply the technology of the present disclosure.

The purpose of the present disclosure is to provide a dual GLP-1/Gcg receptor agonist compound with a longer half-life, to achieve the purpose of dosing once every 2 weeks or once every 4 weeks.

The present disclosure provides a long-acting dual agonist compound and use thereof, which is a dual agonist compound of glucagon-like peptide-1 (GLP-1) receptor and glucagon (Gcg) receptor.

To achieve the above purpose, the present disclosure first provides a compound shown in formula I, a pharmaceutically acceptable salt, solvate, chelate, non-covalent complex or a drug precursor thereof, or any mixture of the above forms.

Formula I
(SEQ ID NO: 6)
His-AA1-Gln-Gly-Thr-Phe-Thr-Ser-Asp-Tyr-Ser-Lys-
Tyr-Leu-Asp-Glu-Lys-Lys-Ala-Lys(R)-Glu-Phe-Val-
Glu-Trp-Leu-Leu-AA2-Gly-Gly-Pro-Ser-Ser-Gly-Ala-
Pro-Pro-Pro-Ser-AA3

AA1 in Formula I is selected from the group consisting of Aib, Acpr, Acp, Acpe and Ach;
AA2 in Formula I is selected from the group consisting of Glu and Ser;
AA3 in Formula I is selected from the group consisting of NH₂ and OH;
R in Formula I is selected from the group consisting of

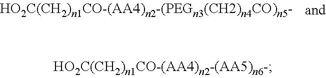

In one embodiment, n1 is an integer selected from 10 to 20;
n2 is an integer selected from 1 to 5;
n3 is an integer selected from 1 to 30;
n4 is an integer selected from 1 to 5;
n5 is an integer selected from 1 to 10; and
n6 is an integer selected from 1 to 10;
AA4 is selected from the group consisting of γGlu, εLys, β-Ala, γ-aminobutyric acid and 5-Ava; and
AA5 is selected from the group consisting of Gly, Ser, Thr, Asp, Glu, Aad, Lys, Orn, Dab and Dap.

The present disclosure further provides a pharmaceutical composition including the compound according to the present disclosure, and provides use of the pharmaceutical composition including the compound according to the present disclosure in the manufacture of a medicament for treating a disease.

In one embodiment, the disease includes type II diabetes, impaired glucose tolerance, type I diabetes, obesity, hypertension, metabolic syndrome, dyslipidemia, cognitive impairment, atherosclerosis, myocardial infarction, coronary heart disease, cardiovascular disease, stroke, inflammatory bowel syndrome and/or dyspepsia or gastric ulcer, liver fibrosis and/or pulmonary fibrosis.

In one embodiment, the present disclosure provides use of the pharmaceutical composition in the manufacture of a medicament for treating type II diabetes with long-lasting efficacy and/or preventing deterioration of type II diabetes.

In one embodiment, the present disclosure provides use of the pharmaceutical composition in the manufacture of a medicament for reducing food intake, reducing β cell apoptosis, increasing pancreatic β cell function, increasing β-cell mass and/or restoring glucose sensitivity of β-cells.

The present disclosure further provides a method for regulating blood sugar in the body, including administering the compound to a subject in need thereof.

More contents of the present disclosure are described in detail below, or some can also be understood from the embodiments of the present disclosure.

Unless otherwise specified, the quantities of different components and reaction conditions used herein could be interpreted as “approximately” or “about” in any case. Accordingly, unless otherwise specified, the numerical parameters cited below and in the claims are approximate parameters, and different numerical parameters may be obtained under respective experimental conditions due to different standard errors.

In the present application, in case there is a disagreement or ambiguity between the chemical formula and the chemical name of a compound, the chemical formula is used to accurately define the compound. The compounds described herein may contain one or more chiral centers, and/or double bonds and the like, and may also exist as stereoisomers, including double bond isomers (such as geometric isomers), optical enantiomers or diastereomers. Accordingly, any chemical structure within the scope of the description herein, whether it contains the above-mentioned similar structure in part or in its entirety, includes all possible enantiomers and diastereomers of the compound, including any single stereoisomer (such as a single geometric isomer, a single enantiomer or a single diastereomer) and any mixture of these isomers. These mixtures of racemic isomers and stereoisomers can also be further separated into their constituent enantiomers or stereoisomers using different resolution techniques or chiral molecular synthesis methods.

The compounds of Formula I include, but are not limited to, optical isomers, racemates and/or other mixtures of these compounds. In the above case, a single enantiomer or diastereomer, such as an optically active isomer, can be obtained by asymmetric synthesis or racemate resolution. The resolution of the racemate can be achieved by various methods, such as conventional recrystallization with a reagent that assists the resolution, or by chromatography. In addition, the compounds of Formula I also include cis and/or trans isomers with double bonds.

The compounds described in the present disclosure include, but are not limited to, the compound represented by Formula I and all of the various pharmaceutically acceptable forms thereof, including various pharmaceutically acceptable salts, solvates, complexes, chelates, non-covalent complexes, prodrugs based on the above substances, and any mixture of the above forms.

The compounds represented by Formula I provided by the present disclosure are stable and are not easily degraded by dipeptidyl peptidase IV (DPP-IV) in the body. They function as Gcg/GLP-I dual agonist analogues with significant effect on lowering blood sugar and reducing body weight.

The present disclosure further provides a production method, including taking Rink Amide MBHA resin as a starting resin, producing a peptide resin using solid phase peptide synthesis method, subjecting the peptide resin to acid hydrolysis to obtain a crude product, and finally purifying the crude product to obtain a pure product; and the step of producing a peptide resin using solid phase peptide synthesis method is performed by sequentially connecting the corresponding protected amino acids or fragments in the sequence to the carrier resin using solid phase coupling synthesis method to obtain the peptide resin.

In the above production method, the amount of the Fmoc-protected amino acid or protected amino acid fragment is 1.2 to 6 times the total molar number of the resin in the reaction; preferably 2.5 to 3.5 times.

In the above production method, the resin substitution is 0.2 to 1.0 mmol/g resin, preferably, the resin substitution is 0.3 to 0.5 mmol/g resin.

Embodiment of the present disclosure, the solid phase coupling synthesis method is performed by removing the Fmoc protecting group from the protected amino acid-resin obtained in the previous step, and then coupling the resulting product with the next protected amino acid; and the removing Fmoc protecting group is performed for 10 to 60 minutes, preferably 15 to 25 minutes; and the coupling is performed for 60 to 300 minutes, preferably 100 to 140 minutes.

A condensation reagent is added in the coupling reaction, which is selected from the group consisting of DIC (N,N-diisopropylcarbodiimide), N,N-dicyclohexylcarbodiimide, benzotriazole-1-yl-oxytripyrrolidinophosphine hexafluorophosphate, 2-(7-aza-1H-benzotriazole-1-yl)-1,1,3,3-tetramethyluronium hexafluorophosphate, benzotriazole-N,N,N′,N′-tetramethyluronium hexafluorophosphate and O-benzotriazole-N,N,N′,N′-tetramethyluronium tetrafluoroborate, preferably N,N-diisopropylcarbodiimide; and the molar number of the condensation reagent is 1.2 to 6 times the total molar number of amino groups in the amino resin, preferably 2.5 to 3.5 times.

An activation reagent is added in the coupling reaction, which is selected from the group consisting of 1-hydroxybenzotriazole and N-hydroxy-7-azabenzotriazole, preferably 1-hydroxybenzotriazole; and the amount of the activation reagent is 1.2 to 6 times the total molar number of amino groups in the amino resin, preferably 2.5 to 3.5 times.

As one embodiment of the present disclosure, the Fmoc-removing reagent is a mixed solution of PIP/DMF (piperidine/N,N-dimethylformamide), and the piperidine content in the mixed solution is 10 to 30% (V); the amount of the Fmoc-removing reagent is 5 to 15 mL per gram of amino resin, preferably 8 to 12 mL per gram of amino resin.

In one embodiment, the peptide resin is subjected to acid hydrolysis to remove the resin and side chain protecting groups to obtain a crude product.

In one embodiment, the acid hydrolysis agent used in the acid hydrolysis of peptide resin is a mixed solvent of trifluoroacetic acid (TFA), 1,2-ethanedithiol (EDT) and water; and the volume ratio of the mixed solvent is 80 to 95% of TFA, 1 to 10% of EDT and water for the balance.

In one embodiment, the volume ratio of the mixed solvent is 89 to 91% of TFA, 4 to 6% of EDT and water for the balance. Optimally, the volume ratio of the mixed solvent is 90% of TFA, 5% of EDT and water for the balance.

The amount of the acid hydrolysis agent used is 4 to 15 mL of acid hydrolysis agent per gram of peptide resin; preferably, the amount of acid hydrolysis agent used is 7 to 10 mL per gram of peptide resin.

The acid hydrolysis is performed for 1 to 6 hours at room temperature, preferably 3 to 4 hours.

Further, the crude product is purified by high performance liquid chromatography and freeze-dried to obtain a pure product.

The terms corresponding to the abbreviations involved in the present disclosure are shown in Table 1:

TABLE 1
Comparison of terms and abbreviations

| Abbreviation | Term | Abbreviation | Term |
|---|---|---|---|
| Fmoc | 9-Fluorenylmethoxy- | OtBu | tert-Butoxy |
| | carbonyl | | |
| tBu | tert-Butyl | Boc | tert-Butoxycarbonyl |
| Trt | Trityl | Pbf | (2,3-dihydro-2,2,4,6,7- |
| | | | pentamethylbenzofuran-5- |
| | | | yl)sulfonyl |
| Ala | Alanine | Leu | Leucine |
| Arg | Arginine | Lys | Lysine |
| Asn | Asparagine | Met | Methionine |
| Asp | Aspartic acid | Phe | Phenylalanine |
| Cys | Cysteine | Pro | Proline |
| Gln | Glutamine | Ser | Serine |
| Glu | Glutamic acid | Thr | Threonine |
| Gly | Glycine | Trp | Tryptophan |
| His | Histidine | Tyr | Tyrosine |
| Ile | Isoleucine | Val | Valine |

The raw materials and reagents used in the production of long-acting dual agonist compound provided by the present disclosure are all commercially available.

The present disclosure is further described below in conjunction with examples:

### Example 1 Production of Compound 1

The sequence of Compound 1 is set forth in SEQ ID NO: 1:

(SEQ ID NO: 1)
His-Aib-Gln-Gly-Thr-Phe-Thr-Ser-Asp-Tyr-Ser-Lys-
Tyr-Leu-Asp-Glu-Lys-Lys-Ala-Lys(AEEA-AEEA-γGlu-
eicosanedioic acid)-Glu-Phe-Val-Glu-Trp-Leu-Leu-
Glu-Gly-Gly-Pro-Ser-Ser-Gly-Ala-Pro-Pro-Pro-Ser-NH₂

### 1. Synthesis of Peptide Resin

Rink Amide BHHA resin was used as a carrier resin, and the protected amino acids shown in the following table were sequentially connected to the resin through removing Fmoc protection and coupling reaction to obtain a peptide resin. The protected amino acids used in this example are shown in Table 2:

TABLE 2
Protected amino acids used in Example 1

| Order of connecting | |
|---|---|
| peptide n= | Protected amino acid |
| 1 | Fmoc-Ser(tBu) |
| 2 | Fmoc-Pro |
| 3 | Fmoc-Pro |
| 4 | Fmoc-Pro |
| 5 | Fmoc-Ala |
| 6 | Fmoc-Gly |
| 7 | Fmoc-Ser(tBu) |
| 8 | Fmoc-Ser(tBu) |
| 9 | Fmoc-Pro |
| 10 | Fmoc-Gly |
| 11 | Fmoc-Gly |
| 12 | Fmoc-Glu(OtBu) |
| 13 | Fmoc-Leu |
| 14 | Fmoc-Leu |
| 15 | Fmoc-Trp(Boc) |
| 16 | Fmoc-Glu(OtBu) |
| 17 | Fmoc-Val |
| 18 | Fmoc-Phe |
| 19 | Fmoc-Glu(OtBu) |
| 20 | Fmoc-Lys(Alloc) |
| 21 | Fmoc-Ala |
| 22 | Fmoc-Lys(Boc) |
| 23 | Fmoc-Lys(Boc) |
| 24 | Fmoc-Glu(OtBu) |
| 25 | Fmoc-Asp(OtBu) |
| 26 | Fmoc-Leu |
| 27 | Fmoc-Tyr(tBu) |
| 28 | Fmoc-Lys(Boc) |
| 29 | Fmoc-Ser(tBu) |
| 30 | Fmoc-Tyr(tBu) |
| 31 | Fmoc-Asp(OtBu) |
| 32 | Fmoc-Ser(tBu) |
| 33 | Fmoc-Thr(tBu) |
| 34 | Boc-Phe |
| 35 | Fmoc-Thr(tBu) |
| 36 | Fmoc-Gly |
| 37 | Fmoc-Gln(Trt) |
| 38 | Fmoc-Aib |
| 39 | Boc-His(Trt) |
| Side chain-1 | Fmoc-AEEA |
| Side chain-2 | Fmoc-AEEA |
| Side chain-3 | Fmoc-γGlu-OtBu |
| Side chain-4 | 20-(tert-Butoxy)-20-oxoicosanoic acid |

### (1) Coupling of the First Protected Amino Acid in the Main Chain

3 mmol of the first protected amino acid and 3 mmol of HOBt were dissolved in an appropriate amount of DMF; 3 mmol of DIC was slowly added into the solution of protected amino acid in DMF under stirring, and the mixture was subjected to reaction at room temperature for 30 min with stirring to obtain an activated protected amino acid solution for standby use.

1 mmol of Rink amide MBHA resin (with a substitution value of about 0.4 mmol/g) was deprotected using 20% PIP/DMF solution for 25 minutes, washed and filtered to obtain a de-Fmoc resin.

The solution containing the activated first protected amino acid was added to the resin without Fmoc, and subjected to coupling reaction for 60 to 300 minutes, filtered and washed to obtain a resin coupling with one protected amino acid.

### (2) Coupling of the 2nd to 39th Protected Amino Acids in the Main Chain

The corresponding 2nd to 39th protected amino acids were sequentially connected to resin using the same method for connecting the first protected amino acid in the main chain to obtain a resin coupling with 39 amino acids of the main chain.

### (3) Coupling of the First Protected Amino Acid in the Side Chain

3 mmol of the first protected amino acid of the side chain and 3 mmol of HOBt were dissolved with an appropriate amount of DMF; 3 mmol of DIC was slowly added to the solution of the protected amino acid in DMF under stirring, and the mixture was subjected to reaction at room temperature for 30 min with stirring to obtain a solution of activated protected amino acid.

2.5 mmol of tetrakistriphenylphosphine palladium and 25 mmol of phenylsilane were dissolved in an appropriate amount of dichloromethane, deprotected for 4 hours, filtered and washed to obtain a resin without Alloc for later use.

The solution containing activated first protected amino acid of the side chain was added to the resin without Alloc, subjected to coupling reaction for 60 to 300 minutes, filtered and washed to obtain a resin coupling with the first protected amino acid of the side chain.

### (4) Coupling of Protected Amino Acid of the Side Chain

The corresponding protected amino acids and single protected fatty acid of the side chain were connected in turn, using the same method for the coupling of the first protected amino acid of the main chain to obtain a peptide resin.

### 2. Production of Crude Product

The above peptide resin was added with a cleavage reagent with a volume ratio of TFA:water:EDT=95:5:5 (cleavage reagent 10 mL/g resin), stirred evenly, and reacted at room temperature for 3 hours. The reaction mixture was filtered with a sand core funnel, and the filtrate was collected. The resin was washed with a small amount of TFA 3 times, and the filtrates were combined, concentrated under reduced pressure, and added with anhydrous ether for precipitation. Then the precipitate was washed with anhydrous ether 3 times, dried by suction to obtain an off-white powder as the crude product.

### 3. Production of Pure Product

The above crude product was added with water, stirred, and adjusted to a pH of 8.0 with ammonia water until completely dissolved. The solution was filtered with a mixed membrane filter with a pore size of 0.45 μm, and purified for later use.

The purification was performed using high performance liquid chromatography, and the packing material of the chromatographic column for purification was reverse phase C18 with a column particle size of 10 μm, the mobile phase system was 0.1% TFA/water solution-0.1% TFA/acetonitrile solution, the flow rate of the 30 mm×250 mm chromatographic column was 20 mL/min, a gradient system elution was adopted, and cyclic loading was used for purification. The solution of the crude product was loaded on the chromatographic column, the mobile phase elution was started, and the main peak was collected and evaporated to remove acetonitrile to obtain a purified intermediate concentrate.

The purified intermediate concentrate was filtered with a 0.45 μm filter membrane for standby use. The salt was exchanged by high performance liquid chromatography. The mobile phase system was 1% acetic acid/water solution-acetonitrile, and the packing material of the chromatographic column for purification was reverse phase C18 with a column particle size of 10 μm. The flow rate of the 30 mm×250 mm chromatographic column was 20 mL/min (the flow rate could be adjusted according to the different specifications of the chromatographic column); a gradient elution and cyclic loading method were used, the sample was loaded into the chromatographic column, the mobile phase elution was started, the spectrum was collected, and the change of absorbance was observed. The main peak fraction of salt exchange was collected, and the purity thereof was detected by analytical liquid phase. The main peak fractions of salt exchange were combined, and concentrated under reduced pressure to obtain an aqueous solution of the pure product in acetic acid, which was freeze-dried to obtain 0.35 g pure product with a purity of 97.9% and a total yield of 7.0%. The molecular weight was 5012.6 (100% M+H).

### Example 2 Production of Compound 2

The sequence of Compound 2 is set forth in SEQ ID NO: 2:

(SEQ ID NO: 2)
His-Aib-Gln-Gly-Thr-Phe-Thr-Ser-Asp-Tyr-Ser-Lys-
Tyr-Leu-Asp-Glu-Lys-Lys-Ala-Lys(PEG5C
H2CO-γGlu-eicosanedioic acid)-Glu-Phe-Val-Glu-Trp-
Leu-Leu-Glu-Gly-Gly-Pro-Ser-Ser-Gly-Ala-Pro-Pro-
Pro-Ser-NH₂

The production method was the same as that of Example 1, and the protected amino acids used are shown in Table 3:

TABLE 3
Protected amino acids used in Example 2

| Order of connecting | |
|---|---|
| peptide n= | Protected amino acid |
| 1 | Fmoc-Ser(tBu) |
| 2 | Fmoc-Pro |
| 3 | Fmoc-Pro |
| 4 | Fmoc-Pro |
| 5 | Fmoc-Ala |
| 6 | Fmoc-Gly |
| 7 | Fmoc-Ser(tBu) |
| 8 | Fmoc-Ser(tBu) |
| 9 | Fmoc-Pro |
| 10 | Fmoc-Gly |
| 11 | Fmoc-Gly |
| 12 | Fmoc-Glu(OtBu) |
| 13 | Fmoc-Leu |
| 14 | Fmoc-Leu |
| 15 | Fmoc-Trp(Boc) |
| 16 | Fmoc-Glu(OtBu) |
| 17 | Fmoc-Val |
| 18 | Fmoc-Phe |
| 19 | Fmoc-Glu(OtBu) |
| 20 | Fmoc-Lys(Alloc) |
| 21 | Fmoc-Ala |
| 22 | Fmoc-Lys(Boc) |
| 23 | Fmoc-Lys(Boc) |
| 24 | Fmoc-Glu(OtBu) |
| 25 | Fmoc-Asp(OtBu) |
| 26 | Fmoc-Leu |
| 27 | Fmoc-Tyr(tBu) |
| 28 | Fmoc-Lys(Boc) |
| 29 | Fmoc-Ser(tBu) |
| 30 | Fmoc-Tyr(tBu) |
| 31 | Fmoc-Asp(OtBu) |
| 32 | Fmoc-Ser(tBu) |
| 33 | Fmoc-Thr(tBu) |
| 34 | Boc-Phe |
| 35 | Fmoc-Thr(tBu) |
| 36 | Fmoc-Gly |
| 37 | Fmoc-Gln(Trt) |
| 38 | Fmoc-Aib |
| 39 | Boc-His(Trt) |
| Side chain-1 | Fmoc-PEG₅CH₂COOH |
| Side chain-2 | Fmoc-γGlu-OtBu |
| Side chain-3 | 20-(tert-Butoxy)-20-oxoicosanoic acid |

0.43 g of pure product was obtained, with a purity of 97.0% and a total yield of 8.6%. The molecular weight was 4999.6 (100% M+H).

### Example 3 Production of Compound 3

The sequence of Compound 3 is set forth in SEQ ID NO: 3:

(SEQ ID NO: 3)
His-Aib-Gln-Gly-Thr-Phe-Thr-Ser-Asp-Tyr-Ser-Lys-
Tyr-Leu-Asp-Glu-Lys-Lys-Ala-Lys(Gly-Gly-Ser-Gly-
Ser-Gly-γGlu-eicosanedioic acid-γGlu-eicosanedioic
acid)-Glu-Phe-Val-Glu-Trp-Leu-Leu-Glu-Gly-Gly-Pro-
Ser-Ser-Gly-Ala-Pro-Pro-Pro-Ser-NH₂

The production method was the same as that of Example 1, and the protected amino acids used are shown in Table 4:

TABLE 4
Protected amino acids used in Example 3

| Order of connecting | |
|---|---|
| peptide n= | Protected amino acid |
| 1 | Fmoc-Ser(tBu) |
| 2 | Fmoc-Pro |
| 3 | Fmoc-Pro |
| 4 | Fmoc-Pro |
| 5 | Fmoc-Ala |
| 6 | Fmoc-Gly |
| 7 | Fmoc-Ser(tBu) |
| 8 | Fmoc-Ser(tBu) |
| 9 | Fmoc-Pro |
| 10 | Fmoc-Gly |
| 11 | Fmoc-Gly |
| 12 | Fmoc-Glu(OtBu) |
| 13 | Fmoc-Leu |
| 14 | Fmoc-Leu |
| 15 | Fmoc-Trp(Boc) |
| 16 | Fmoc-Glu(OtBu) |
| 17 | Fmoc-Val |
| 18 | Fmoc-Phe |
| 19 | Fmoc-Glu(OtBu) |
| 20 | Fmoc-Lys(Alloc) |
| 21 | Fmoc-Ala |
| 22 | Fmoc-Lys(Boc) |
| 23 | Fmoc-Lys(Boc) |
| 24 | Fmoc-Glu(OtBu) |
| 25 | Fmoc-Asp(OtBu) |
| 26 | Fmoc-Leu |
| 27 | Fmoc-Tyr(tBu) |
| 28 | Fmoc-Lys(Boc) |
| 29 | Fmoc-Ser(tBu) |
| 30 | Fmoc-Tyr(tBu) |
| 31 | Fmoc-Asp(OtBu) |
| 32 | Fmoc-Ser(tBu) |
| 33 | Fmoc-Thr(tBu) |
| 34 | Boc-Phe |
| 35 | Fmoc-Thr(tBu) |
| 36 | Fmoc-Gly |
| 37 | Fmoc-Gln(Trt) |
| 38 | Fmoc-Aib |
| 39 | Boc-His(Trt) |
| Side chain-1 | Fmoc-Gly |
| Side chain-2 | Fmoc-Gly |
| Side chain-3 | Fmoc-Ser(tBu) |
| Side chain-4 | Fmoc-Gly |
| Side chain-5 | Fmoc-Ser(tBu) |
| Side chain-6 | Fmoc-Gly |
| Side chain-7 | Fmoc-γGlu-OtBu |
| Side chain-8 | 20-(tert-Butoxy)-20-oxoicosanoic acid |

0.33 g of pure product was obtained with a purity of 93.4% and a total yield of 6.4%. The molecular weight was 5124.6 (100% M+H).

### Example 4 Production of Compound 4

The sequence of Compound 4 is set forth in SEQ ID NO: 4:

(SEQ ID NO: 4)
His-Aib-Gln-Gly-Thr-Phe-Thr-Ser-Asp-Tyr-Ser-Lys-
Tyr-Leu-Asp-Glu-Lys-Lys-Ala-Lys(Gly-Gly-Glu-Gly-
Glu-Gly-γGlu-eicosanedioic acid)-Glu-Phe-Val-Glu-
Trp-Leu-Leu-Glu-Gly-Gly-Gly-Pro-Ser-Ser-Gly-Ala-
Pro-Pro-Pro-Ser-NH₂

The production method was the same as that of Example 1, and the protected amino acids used are shown in Table 5:

TABLE 5
Protected amino acids used in Example 4

| Order of connecting | |
|---|---|
| peptide n= | Protected amino acid |
| 1 | Fmoc-Ser(tBu) |
| 2 | Fmoc-Pro |
| 3 | Fmoc-Pro |
| 4 | Fmoc-Pro |
| 5 | Fmoc-Ala |
| 6 | Fmoc-Gly |
| 7 | Fmoc-Ser(tBu) |
| 8 | Fmoc-Ser(tBu) |
| 9 | Fmoc-Pro |
| 10 | Fmoc-Gly |
| 11 | Fmoc-Gly |
| 12 | Fmoc-Glu(OtBu) |
| 13 | Fmoc-Leu |
| 14 | Fmoc-Leu |
| 15 | Fmoc-Trp(Boc) |
| 16 | Fmoc-Glu(OtBu) |
| 17 | Fmoc-Val |
| 18 | Fmoc-Phe |
| 19 | Fmoc-Glu(OtBu) |
| 20 | Fmoc-Lys(Alloc) |
| 21 | Fmoc-Ala |
| 22 | Fmoc-Lys(Boc) |
| 23 | Fmoc-Lys(Boc) |
| 24 | Fmoc-Glu(OtBu) |
| 25 | Fmoc-Asp(OtBu) |
| 26 | Fmoc-Leu |
| 27 | Fmoc-Tyr(tBu) |
| 28 | Fmoc-Lys(Boc) |
| 29 | Fmoc-Ser(tBu) |
| 30 | Fmoc-Tyr(tBu) |
| 31 | Fmoc-Asp(OtBu) |
| 32 | Fmoc-Ser(tBu) |
| 33 | Fmoc-Thr(tBu) |
| 34 | Boc-Phe |
| 35 | Fmoc-Thr(tBu) |
| 36 | Fmoc-Gly |
| 37 | Fmoc-Gln(Trt) |
| 38 | Fmoc-Aib |
| 39 | Boc-His(Trt) |
| Side chain-1 | Fmoc-Gly |
| Side chain-2 | Fmoc-Gly |
| Side chain-3 | Fmoc-Glu(OtBu) |
| Side chain-4 | Fmoc-Gly |
| Side chain-5 | Fmoc-Glu(OtBu) |
| Side chain-6 | Fmoc-Gly |
| Side chain-7 | Fmoc-γGlu-OtBu |
| Side chain-8 | 20-(tert-Butoxy)-20-oxoicosanoic acid |

0.30 g of pure product was obtained with a purity of 96.5% and a total yield of 5.8%. The molecular weight was 5208.7 (100% M+H).

### Example 5 Synthesis of Control Compound

The control compound is OXM3, and its amino acid sequence is set forth in SEQ ID NO: 5:

(SEQ ID NO: 5)
His-Aib-Gln-Gly-Thr-Phe-Thr-Ser-Asp-Tyr-Ser-Lys-
Tyr-Leu-Asp-Glu-Lys-Lys-Ala-Lys(AEEA-AEEA-
γGlu-eicosanedioic acid)-Glu-Phe-Val-Glu-
Trp-Leu-Leu-Glu-Gly-Gly-Pro-Ser-Ser-Gly-NH₂

The production method was the same as in Example 1, and the molecular weight was 4563.1.

### Example 6 Determination of GLP-1 Activity

### 1. Determination Method

GLP-1R activates the intracellular adenylate cyclase pathway under the stimulation of its specific agonist, to increase the cAMP level and ultimately lead to the production and release of insulin. The stable cell line expressing GLP-1R was stimulated by the test substance to rapidly increase the intracellular cAMP level. The relative light units (RLU) in the cells after stimulation by each dose of the test substance were determined by the chemiluminescence method, and then the EC50 of the agonist was calculated. This activity determination method is currently a common determination method for GLP-1 receptor agonist activity at home and abroad.

A CHO-K1 cell line that stably expresses GLP-IR was adopted, and the cells were stimulated with different concentrations of agonists. The relative light units after stimulation of the cells by each dose of agonists were measured. Using OXM3 as a reference substance, the relative biological activity of each agonist was obtained.

### 2. Determination Results

The determination results are shown in Table 6.

TABLE 6
GLP activity determination

| Compound | Relative biological activity |
|---|---|
| Reference | OXM3 |
| Compound 1 | 188.5% |
| Compound 2 | 259.9% |
| Compound 3 | 158.1% |
| Compound 4 | 115.6% |

The test results show that the GLP-1 activity of the new GIP/GLP-I dual agonist analogue is increased by 15% to 159% compared with OXM3.

### Example 7 Gcg Activity Determination

### 1. Determination Method

GcgR activates the intracellular adenylate cyclase pathway under the stimulation of its specific agonist, to increase the cAMP level and ultimately lead to the production and release of insulin. The stably cell line expressing GcgR was stimulated by the test substance to rapidly increase the intracellular cAMP level. The relative light units (RLU) in the cells after the stimulation by each dose of agonists were determined by the chemiluminescence method, and then the EC50 of the agonist was calculated. This activity determination method is currently a common determination method for Geg receptor agonist activity at home and abroad.

A CHO-K1 cell line stably expressing GcgR was adopted. The cells were stimulated with different concentrations of agonists, and the relative light units (RLU) after the stimulation of cells by each dose of agonists were determined. Using OXM3 as a reference, the relative biological activity of the agonist was obtained.

### 2. Determination Results

The determination results are shown in Table 7.

TABLE 7
Gcg activity determination

| Compound | Biological activity (%) |
|---|---|
| Reference | OXM3 |
| Compound 1 | 90.5% |
| Compound 2 | 86.9% |
| Compound 3 | 94.2% |
| Compound 4 | 49.6% |

The determination results show that the Geg activity of the new GIP/GLP-I dual agonist 1 to 3 is similar to the activity of OXM3.

### Example 8 Determination of Preliminary Pharmacokinetic Properties

The experimental animals were cynomolgus monkeys, and two male cynomolgus monkeys in each group were subcutaneously administrated the compound at a dose of 0.2 mg/kg. The venous blood samples were collected before drug administration (0 h), and 1 h, 2 h, 3 h, 4 h, 8 h, 12 h, 18 h, 24 h, 48 h, 96 h, 144 h and 168 h after administration, respectively. The plasma samples were separated by centrifugation. The blood drug concentrations of the corresponding compounds in the plasma samples were determined by liquid chromatography-mass spectrometry. The half-life of the compound given via subcutaneous (SC) administration is shown in Table 8.

TABLE 8
Determination of preliminary pharmacokinetic properties

| Compound | t_{1/2} (h) |
|---|---|
| Compound 2 | 140.2 |

The modified compound with the highest biological activity (Compound 2) was detected for its pharmacokinetic properties. The result showed that the half-life of Compound 2 was 140.2 hrs, which was converted to a human half-life of about 280 hrs, much higher than the half-life of OXM3 (120 hrs).

The above is a detailed description of a long-acting dual agonist compound provided by the present disclosure. Specific examples are used herein to illustrate the principles and embodiments of the present disclosure. The description of the above examples is only used to help understand the method of the present.

1. A compound, comprising:
(I) an amino acid sequence set forth in Formula I,
Formula I
(SEQ ID NO: 6)
His-AA1-Gln-Gly-Thr-Phe-Thr-Ser-Asp-Tyr-Ser-Lys-
Tyr-Leu-Asp-Glu-Lys-Lys-Ala-Lys(R)-Glu-Phe-Val-
Glu-Trp-Leu-Leu-AA2-Gly-Gly-Pro-Ser-Ser-Gly-Ala-
Pro-Pro-Pro-Ser-AA3
AA1 in Formula I is selected from the group consisting of Aib, Acpr, Acp, Acpe and Ach;
AA2 in Formula I is selected from the group consisting of Glu and Ser;
AA3 in Formula I is selected from the group consisting of NH₂ and OH;
R in Formula I is selected from the group consisting of HO₂C(CH₂)ₙ₁CO-(AA4)ₙ₂-(PEGₙ₃(CH2)ₙ₄CO)ₙ₅- and HO₂C(CH₂)ₙ₁CO-(AA4)ₙ₂-(AA5)ₙ₆-;
wherein, AA4 is selected from the group consisting of γGlu, εLys, β-Ala, γ-aminobutyric acid and 5-Ava; and
AA5 is selected from the group consisting of Gly, Ser, Thr, Asp, Glu, Aad, Lys, Orn, Dab and Dap; or
(II) an amino acid sequence obtained from substitution, deletion, addition and/or replacement of one or more amino acids of the amino acid sequence set forth in (I); or
(III) a sequence with more than 90% homology to the amino acid sequence set forth in (I); or
(IV) a pharmaceutically acceptable salt, solvate, chelate or non-covalent complex of the compound shown in Formula I; and/or
(V) a drug precursor based on the compound shown in Formula I; and/or
(VI) a mixture comprising (I), (II), (III), (IV) and/or (V).

2. The compound according to claim 1, wherein:
n1 is an integer selected from 10 to 20;
n2 is an integer selected from 1 to 5;
n3 is an integer selected from 1 to 30;
n4 is an integer selected from 1 to 5;
n5 is an integer selected from 1 to 10; and
n6 is an integer selected from 1 to 10.

3. A method for producing the compound according to claim 1, comprising:
Step 1, performing solid-phase polypeptide synthesis to obtain a peptide resin; and
Step 2, performing acid hydrolysis and purification to obtain the compound.

4. (canceled)

5. A method for preventing and/or treating a disease comprising administering the compound according to claim 1 to the subject in need thereof.

6. The method according to claim 5, wherein the disease is selected from the group consisting of:
type II diabetes, impaired glucose tolerance, type I diabetes, obesity, hypertension, metabolic syndrome, dyslipidemia, cognitive impairment, atherosclerosis, myocardial infarction, coronary heart disease, cardiovascular disease, stroke, inflammatory bowel syndrome and/or dyspepsia or gastric ulcer, liver fibrosis and pulmonary fibrosis.

7. A method for treating type II diabetes with long-lasting efficacy and/or preventing deterioration of type II diabetes comprising administering the compound according to claim 1 to the subject in need thereof.

8. A method for regulating blood sugar in the body comprising administering the compound according to claim 1 to the subject in need thereof,
wherein, the regulation of blood sugar in the body includes reducing food intake, reducing β cell apoptosis, increasing pancreatic β cell function, increasing β-cell mass and/or restoring the sensitivity of β-cell to glucose.

9. A drug or drug combination, comprising the compound according to claim 1.

10. A method for regulating blood sugar in the body, comprising administering
the drug or drug combination according to claim 9.

11. The compound according to claim 1, wherein the compound is selected from the group consisting of:
(SEQ ID NO: 1)
His-Aib-Gln-Gly-Thr-Phe-Thr-Ser-Asp-Tyr-Ser-Lys-Tyr-Leu-Asp-Glu-Lys-Lys-
Ala-Lys(AEEA-AEEA-γGlu-eicosanedioic acid)-Glu-Phe-Val-Glu-Trp-Leu-Leu-
Glu-Gly-Gly-Pro-Ser-Ser-Gly-Ala-Pro-Pro-Pro-Ser-NH2,
(SEQ ID NO: 2)
His-Aib-Gln-Gly-Thr-Phe-Thr-Ser-Asp-Tyr-Ser-Lys-Tyr-Leu-Asp-Glu-Lys-Lys-
Ala-Lys(PEG5CH2CO-γGlu-eicosanedioic acid)-Glu-Phe-Val-Glu-Trp-Leu-Leu-
Glu-Gly-Gly-Pro-Ser-Ser-Gly-Ala-Pro-Pro-Pro-Ser-NH2,
(SEQ ID NO: 3)
His-Aib-Gln-Gly-Thr-Phe-Thr-Ser-Asp-Tyr-Ser-Lys-Tyr-Leu-Asp-Glu-Lys-Lys-
Ala-Lys(Gly-Gly-Ser-Gly-Ser-Gly-γGlu-eicosanedioic acid-γGlu-eicosanedioic
acid)-Glu-Phe-Val-Glu-Trp-Leu-Leu-Glu-Gly-Gly-Pro-Ser-Ser-Gly-Ala-Pro-Pro-
Pro-Ser-NH2,
and
(SEQ ID NO: 4)
His-Aib-Gln-Gly-Thr-Phe-Thr-Ser-Asp-Tyr-Ser-Lys-Tyr-Leu-Asp-Glu-Lys-Lys-
Ala-Lys(Gly-Gly-Glu-Gly-Glu-Gly-γGlu-eicosanedioic acid)-Glu-Phe-Val-Glu-
Trp-Leu-Leu-Glu-Gly-Gly-Gly-Pro-Ser-Ser-Gly-Ala-Pro-Pro-Pro-Ser-NH2.


SEQUENCE LISTING (6 sequences)

SEQ ID NO: 1 - protein, 39 aa, synthetic construct
  MOD_RES 20: note = Lysine is modified with AEEA-AEEA-gamma Glu-eicosanedioic acid
  MOD_RES 2: note = X at location 2 is Aib
HXQGTFTSDYSKYLDEKKAKEFVEWLLEGGPSSGAPPPS

SEQ ID NO: 2 - protein, 39 aa, synthetic construct; identical to SEQ ID NO: 1
  MOD_RES 20: note = Lysine is modified with PEG5CH2CO-gamma Glu-eicosanedioic acid
  MOD_RES 2: note = X at location 2 is Aib

SEQ ID NO: 3 - protein, 39 aa, synthetic construct; identical to SEQ ID NO: 1
  MOD_RES 20: note = Lysine is modified with Gly-Gly-Ser-Gly-Ser-Gly-gamma Glu-eicosanedioic acid-gamma Glu-eicosanedioic acid
  MOD_RES 2: note = X at location 2 is Aib

SEQ ID NO: 4 - protein, 39 aa, synthetic construct; identical to SEQ ID NO: 1
  MOD_RES 20: note = Lysine is modified with Gly-Gly-Glu-Gly-Glu-Gly-gamma Glu-eicosanedioic acid
  MOD_RES 2: note = X at location 2 is Aib

SEQ ID NO: 5 - protein, 34 aa, synthetic construct
  MOD_RES 20: note = Lysine is modified with AEEA-AEEA-gamma Glu-eicosanedioic acid
  MOD_RES 2: note = X at location 2 is Aib
HXQGTFTSDYSKYLDEKKAKEFVEWLLEGGPSSG

SEQ ID NO: 6 - protein, 39 aa, synthetic construct
  VAR_SEQ 2: note = Amino acid at location 2 is selected from the group consisting of Aib, Acpr, Acp, Acpe and Ach.
  MOD_RES 20: note = Lysine is modified with R, and R is HO2C(CH2)n1CO-(AA4)n2-(PEGn3(CH2)n4CO)n5- or HO2C(CH2)n1CO-(AA4)n2-(AA5)n6-, wherein AA4 is selected from the group consisting of gamma-Glu, epsilon-Lys, beta-Ala, gamma-aminobutyric acid and 5-Ava, AA5 is selected from the group consisting of Gly, Ser, Thr, Asp, Glu, Aad, Lys, Orn, Dab and Dap. n1 is an integer selected from 10 to 20, n2 is an integer selected from 1 to 5, n3 is an integer selected from 1 to 30, n4 is an integer selected from 1 to 5, n5 is an integer selected from 1 to 10 and n6 is an integer selected from 1 to 10.
  VAR_SEQ 28: note = Amino acid at location 28 is Glu or Ser.
  MOD_RES 39: note = Amino acid at location 39 is linked with NH2 or OH
HXQGTFTSDYSKYLDEKKAKEFVEWLLXGGPSSGAPPPS